(19) United States
(12) Patent Application Publication
(10) Pub. No.: US 2022/0238190 A1
(43) Pub. Date: Jul. 28, 2022

(54) SYSTEMS AND METHOD FOR TARGETED MOLECULAR DESIGN

(71) Applicant: Phronesis Artificial Intelligence, Inc., Daphne, AL (US)

(72) Inventor: William Carl SPAGNOLI, Marina Del Rey, CA (US)

(21) Appl. No.: 17/584,053

(22) Filed: Jan. 25, 2022

Related U.S. Application Data
(60) Provisional application No. 63/142,074, filed on Jan. 27, 2021.

(51) Int. Cl. G16C 20/50; G16B 15/30

(52) U.S. Cl. CPC G16C 20/50; G16B 15/30

(57) ABSTRACT

Systems, devices, and methods for an iterative process for targeted molecular design comprising: adding one or more head starthead start molecules to a molecular database; measuring the added one or more head starthead start molecules in one or more metrics; adding the measured one or more head starthead start molecules to a master results table; assigning one or more scores for each secondary metric goal to the one or more head starthead start molecules in the master results table; selecting one or more head starthead start molecules based on the assigned scores for each metric and a random selection from the one or more head starthead start molecules; training a model using the selected one or more head start molecules and generating one or more new molecules based on the trained model.

### CROSS-REFERENCE TO RELATED APPLICATIONS

This application claims benefit of U.S. provisional patent application Ser. No. 63/142,074, filed Jan. 27, 2021, which is herein incorporated by reference.

### FIELD

Embodiments relate generally to molecular design, and more particularly to automated targeted molecular design.

There exists a need to discover molecules capable of use for many applications, and in particular, as candidates for the prevention or treatment of disease, including infectious diseases. For example, viruses are known to attach to, and infect, cells by the connection of a cell ligand to virus receptor. The receptor mimics some other beneficial connection with the cell, and is thus able to attach to the cell and use the cell to replicate itself. To prevent the virus from accomplishing this, a means of blocking the virus receptor so that it cannot attach to the cell can be used.

One measure of such a molecule attaching to a receptor of a virus is known as binding affinity, and is one of the key factors of whether a molecule will become attached to a target receptor in a virus. However, other secondary properties of the molecule and the receptor are important to an understanding of the likelihood that a molecule could be a candidate for effectively blocking a target receptor of a virus, for example the molecules molecular weight, its solubility in bodily fluids, and other factors.

### SUMMARY

A method embodiment may include: adding one or more head start molecules to a molecular database; measuring the added one or more head start molecules with respect to one or more primary factors or metrics by which the molecules are to be evaluated; adding the measured one or more head start molecules to a master results table; assigning one or more scores for each secondary metric or factor goal for which the molecules are to be evaluated to the one or more head start molecules in the master results table; selecting one or more head start molecules based on the assigned scores for each metric and a random selection from the one or more head start molecules; training a model using the selected one or more head start molecules; and generating one or more new molecules based on the trained model.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1 depicts a top-level functional block diagram of a computing system environment;

FIG. 2 depicts components in communication with a processor of the computing system of FIG. 1;

FIG. 3 depicts a welcome screen of a computing device of the computing system of FIG. 1;

FIG. 4 depicts a settings page of the computing device of FIG. 3;

FIG. 5 depicts a receptor selection page of the computing device of FIG. 3;

FIG. 6 depicts an optional settings page of the computing device of FIG. 3;

FIG. 7 depicts a summary page of the computing device of FIG. 3;

FIG. 8 depicts a progress bar page of the computing device of FIG. 3;

FIG. 9 depicts a flow chart of an iterative process for targeted molecular design;

FIG. 10 depicts an output folder of the computing device of FIG. 3;

FIG. 11 depicts an output file associated with the output folder of FIG. 10;

FIG. 12 depicts an output table associated with the output file of FIG. 11;

FIG. 13 depicts a molecular image;

FIG. 14 depicts an alternative molecular image;

FIG. 15 depicts a docking folder;

FIG. 16 depicts a flow diagram of a molecular representation;

FIG. 17 depicts a flow diagram of a neural network generation process;

FIG. 18 depicts a flow diagram of a model training process

FIG. 19 depicts a flow diagram of a system application overview;

FIG. 20 depicts a block diagram of the system of FIG. 19;

FIG. 21 shows a high-level block diagram and process of a computing system for implementing an embodiment of the system and process;

FIG. 22 shows a block diagram and process of an exemplary system in which an embodiment may be implemented; and

FIG. 23 depicts a cloud computing environment for implementing an embodiment of the system and process disclosed herein.

### DETAILED DESCRIPTION

The described technology concerns one or more methods, systems, apparatuses, and mediums storing processor-executable process steps of automated targeted molecular design allowing a user or users to design molecules of any desired traits, and providing detailed metrics for the new molecules to the user or users. In one embodiment, a targeted molecular design application may automatically provide organized, easy to understand, and sortable measurements of newly generated molecules, allowing the user to immediately view side-by-side comparisons of the relevant properties in new molecules. Advantageously, the user sets the parameters of at least two the molecule properties, and resultantly receives one or more molecule designs that are raked against their molecule properties vis a vis the user selected molecule parameters. Thus, where the user selects molecular features that relate to the intended use of the molecule, molecules are generated that inherently possess desired features related to the potential use thereof. Additionally, a molecular representation can be generated, and displayed, to the user.

The techniques introduced below may be implemented by programmable circuitry programmed or configured by software and/or firmware, or entirely by special-purpose circuitry, or in a combination of such forms. Such special-purpose circuitry (if any) can be in the form of, for example, one or more application-specific integrated circuits (ASICs), programmable logic devices (PLDs), field-programmable gate arrays (FPGAs), etc.

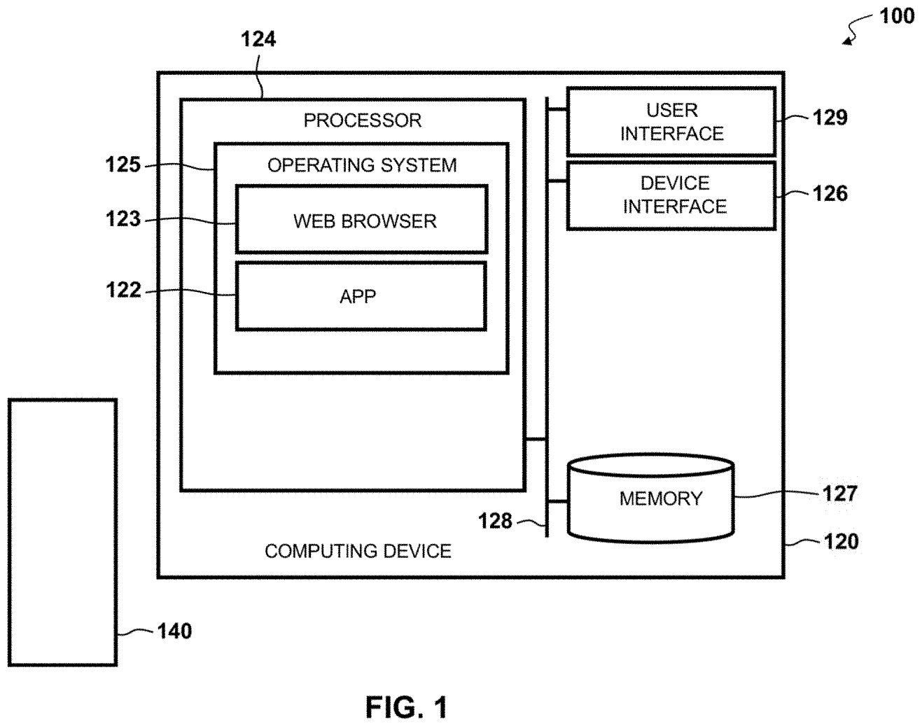

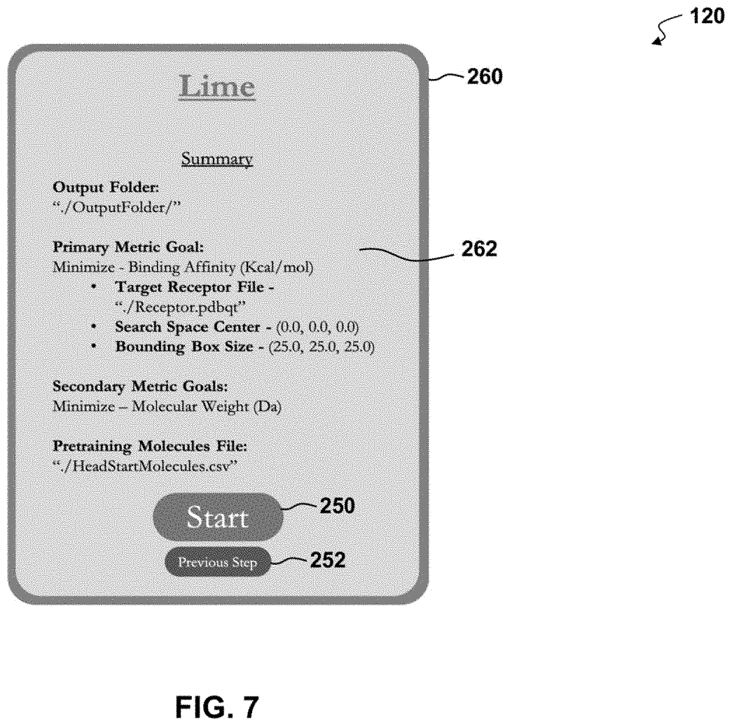

FIGS. 1-18 and the following discussion provide a brief, general description of a suitable computing environment in which aspects of the described technology may be implemented. Although not required, aspects of the technology may be described herein in the general context of computer-executable instructions, such as routines executed by a general- or special-purpose data processing device (e.g., a server or client computer). Aspects of the technology described herein may be stored or distributed on tangible computer-readable media, including magnetically or optically readable computer discs, hard-wired or preprogrammed chips (e.g., EEPROM semiconductor chips), nanotechnology memory, biological memory, or other data storage media. Alternatively, computer-implemented instructions, data structures, screen displays, and other data related to the technology may be distributed over the Internet or over other networks (including wireless networks) on a propagated signal on a propagation medium (e.g., an electromagnetic wave, a sound wave, etc.) over a period of time. In some implementations, the data may be provided on any analog or digital network (e.g., packet-switched, circuit-switched, or other scheme).

The described technology may also be practiced in distributed computing environments where tasks or modules are performed by remote processing devices, which are linked through a communications network, such as a Local Area Network (“LAN”), Wide Area Network (“WAN”), or the Internet. In a distributed computing environment, program modules or subroutines may be located in both local and remote memory storage devices. Those skilled in the relevant art will recognize that portions of the described technology may reside on a server computer, while corresponding portions may reside on a client computer (e.g., PC, mobile computer, tablet, or smartphone). Data structures and transmission of data particular to aspects of the technology are also encompassed within the scope of the described technology.

Present embodiments provide for targeted molecular design wherein a user may be presented with newly-designed molecules that are automatically organized, easy to understand, along with sortable measurements thereof, allowing the user to immediately view side-by-side comparisons of all relevant properties in the newly-designed molecules. In one embodiment, “Fully Autonomous Molecular Evolution” (FAME) may execute a program to continuously measure all newly generated molecules, strategically select the top molecules based on the closeness of their properties as a fit to the desired molecule properties, for example those generated molecules having the lowest binding affinity value, i.e., for example, the lowest K_{D} value, where K_{D} is one measure of equilibrium disassociation constant (and thus the highest likelihood to bind to the receptor), those having the lowest molecular weight, etc., and use these molecules and their properties to continuously retrain itself to create molecules having better binding affinity values, lower molecular weight, etc. More specifically, the top molecules may be chosen for training based upon a user's selected metric goals. For example, if a user selects “Binding Affinity” as the primary molecule metric goal, and “Molecular Weight” as the secondary molecule metric goal, then a plurality of molecules with the top Binding Affinity score may be selected, along with a plurality of molecules with the highest “Weight-Adjusted Binding Affinity Score”, a plurality of molecules with the highest “Similarity-Adjusted Binding Affinity Score” (to ensure diversity), and a plurality of randomly generated molecules from a baseline long short-term memory “LSTM” Molecule Generator network (or other form of sequence-generating neural network) in order to introduce random mutation. For example, weight adjusted binding affinity score is a value assigned to a molecule, relative to other molecules, wherein the molecular weight is considered in addition to the binding affinity in valuing the closeness of the molecule to the desired molecule properties. Similarly, similarity adjusted binding affinity score is a value assigned to a molecule, relative to other molecules, wherein the similarity of the molecule to other generated molecules is considered in addition to the binding affinity in valuing the closeness of the molecule to the desired molecule properties. The higher the score, the less similar the molecule is to other generated molecules For example, if a user selects “Binding Affinity” as the primary molecule metric goal, and “Molecular Weight” as the secondary molecule metric goal, the 35 molecules with the top Binding Affinity score will be selected, along with 5 molecules with the highest “Weight-Adjusted Binding Affinity Score”, 5 molecules with the highest “Similarity-Adjusted Binding Affinity Score” (to ensure diversity), and 5 randomly generated molecules from a baseline LSTM Molecule Generator network in order to introduce random mutation. The number of molecules selected from each metric score, and the metric scores used are dependent upon molecule metric goals selected by the users. Additionally, the number of selected molecules may be larger or smaller with the same relative ratio therebetween, or the relative ratio of each class of molecules can change, or both. Using these scored molecules, and the interrelationship of the score to more than one molecular property, the FAME program can continuously improve its identification of more closely meeting the target molecule metrics for newly developed molecules, and thus generate better molecules for the user's desired goal. This may be achieved without requiring the user to manually score hundreds or thousands of molecules for the FAME system to reference to determine the likelihood a new molecule is a better fit than another molecule to the desired molecule metrics. Manually scoring requires significant time, domain knowledge, and additional software, dramatically increasing design complexity and design cost.

The targeted molecular design system (FAME system) provides an easy-to-use user interface, which allows artificial intelligence (AI) molecular design to be used by researchers in any industry, not only limited to software developers. As such, the targeted molecular design system may be accessible to anyone who needs it, regardless of technological expertise.

The robust targeting algorithm of the targeted molecular design system provides enhanced control over molecular design. For example, when used for drug discovery, the user may want a molecule that not only has a sufficiently low binding affinity value K_{D} (and thus high likelihood to bind) with a target pathogen such as a target receptor of a virus, but also can be administered orally and is simple to synthesize. Thus, here the user would select metrics based on ease of synthesis and molecular weight, as well as binding affinity.

Alternatively, a non-medical user of the FAME system may wish to target molecules having specific pH levels or a specific molecular weight for use in, for example, an industrial process. The targeted molecular design system provides the user with a robust ability to choose a variety of molecular attributes or qualities that the user may wish to have present in a molecule created or designed by the molecular design system. In other embodiments, the targeted molecular design system may provide for new targeting functions and associated target properties of the molecules to be easily added by a user.

The targeted molecular design system addresses a variety of problems across different fields that require an understanding of a diverse collection of fields. For example, for molecules for medical or pharmaceutical applications, the targeted molecular design system not only provides for identifying and designing molecules having optimized binding affinity to a target such as a target receptor, but also has the domain knowledge of the pharmaceutical industry, drug discovery process, and FDA regulations/barriers to drug approval embedded therein or accessible thereto. For other applications, the molecular design system can include in its domain knowledge the application specific metrics for the application, for example, industrial requirements on the storage and shelf life, as well as interactions of the molecule in a process setting, required for a molecule in that particular application.

Therefore, when used in the medical or pharmaceutical field, the targeted molecular design system may design for the needed and required attributes for simultaneously targeting other ideal drug qualities. In the same manner, the targeting of desired attributes for industrial/chemical compounds requires additional domain knowledge of chemistry and material science, which the targeted molecular design system possesses.

It is understood that while molecules with strong-binding affinity to a specified target receptor (low K_{D} value) are a good start for discovering a candidate drug, strong-binding affinity is only one of many necessary molecular qualities for effective drugs.

For example, Remdisivir® has shown great potential as a candidate drug for COVID-19 throughout the current global pandemic due to its binding affinity to the virus' ACE2 receptor, but presents challenges in the production of an adequate global supply due to the complexity required to synthesize the molecule. Additionally, high-quality drug candidates must not have adverse interactions with other drugs and/or the human or other body treated therewith, for example mammalian, reptilian, etc. bodies, be able to permeate through the necessary body membranes for absorption thereof into the body, preferably be soluble enough to be orally administered (for patient acceptance), and meet many more requirements. The present embodiments provide for a system that may not only target strong-binding affinity molecules and other desired molecule traits, but also provide information regarding adverse interactions with other drugs and other pertinent information, such as FDA requirements related to the fabrication, suitability for use, and testing of a newly designed molecule.

Additionally, a user-friendly interface of the targeted molecular design system (FAME system) provides for easy operation for the generation of newly designed molecules with user desired traits for non-tech-savvy users, allowing for widespread adoption thereof across industries.

The targeted molecular design system provides enhanced efficiency in the molecular design process as compared to prior methodologies, where molecular design is an essential process for a wide range of fields, including, but not limited to, drug discovery, industrial material design, chemical innovation, and many more fields. The previous inefficiency in the molecular design process is due to the vast complexity of molecule design and inter-atom and other molecular interactions within the molecule, interaction with other molecules and interaction with other multi-atom structures such as target receptors of a virus, etc. There are estimated to be between 10⁶⁰ and 10⁸⁰ unique molecules currently in existence, with only an estimated 60 Million currently known, documented molecules. The targeted molecular design system may efficiently probe the vast universe of possible molecules, greatly speeding up the design and discovery of new molecules with desired traits. For example, in drug discovery, the current system for narrowing down the potential vast number of potential drug molecules to the top 250 candidate drugs to take to clinical trials typically may take anywhere from 4-7 years, requiring hundreds of millions of dollars and entire teams of expert developers. The targeted molecular design system (FAME system) hereof may remove many of the current barriers to determining which, among many, molecule candidates may have the attributes capable of potentially solving a medical, industrial or other problem, and provides for all forms of molecular design, from drug discovery to chemical compound design, using a quick and easy interface with little to no drug or molecule design experience required.

The present embodiments not only assist in the field of drug discovery, but they also provide algorithms able to solve many of humanity's needs for new molecules. For example, society needs a solution that will provide a stronger new metal alloy able to save a child in a car crash, a new chemical or chemical agent to light exit signs in the dark to avoid radiation exposure from the slightly radioactive paint used in present exit signs, and countless other molecules that offer the potential to save, or enhance, lives.

The present embodiments hereof provide for a simple, user-friendly system that makes targeted molecular design state-of-the-art technology accessible to everyone, regardless of experience.

FIG. 1 illustrates an example of a top-level functional block diagram of a computing system embodiment 100. The example operating environment is shown with a server computer 140 and a computing device 120 comprising:

a processor 124, such as a central processing unit (CPU) or a graphics processing unit (GPU);

addressable memory 127;

an external device interface 126, e.g., an optional universal serial bus port and related processing, and/or an Ethernet port and related processing, and;

an optional user interface 129, e.g., an array of status lights and one or more toggle switches, and/or a display, and/or a keyboard and/or a pointer-mouse system and/or a touch screen.

Optionally, the addressable memory may include any type of computer-readable media that can store data accessible by the computing device 120, such as magnetic hard and floppy disk drives, optical disk drives, magnetic cassettes, tape drives, flash memory cards, digital video disks (DVDs), Bernoulli cartridges, RAMs, ROMs, smart cards, etc. Indeed, any medium for storing or transmitting computer-readable instructions and data may be employed, including a connection port to or node on a network, such as a LAN, WAN, or the Internet. These elements may be in communication with one another via a data bus 128. In some embodiments, via an operating system 125 such as one supporting a web browser 123 and applications 122, the processor 124 may be configured to execute steps of a process establishing a communication channel and processing according to the embodiments described above.

In one embodiment, an application 122 is a targeted molecular design application as described below.

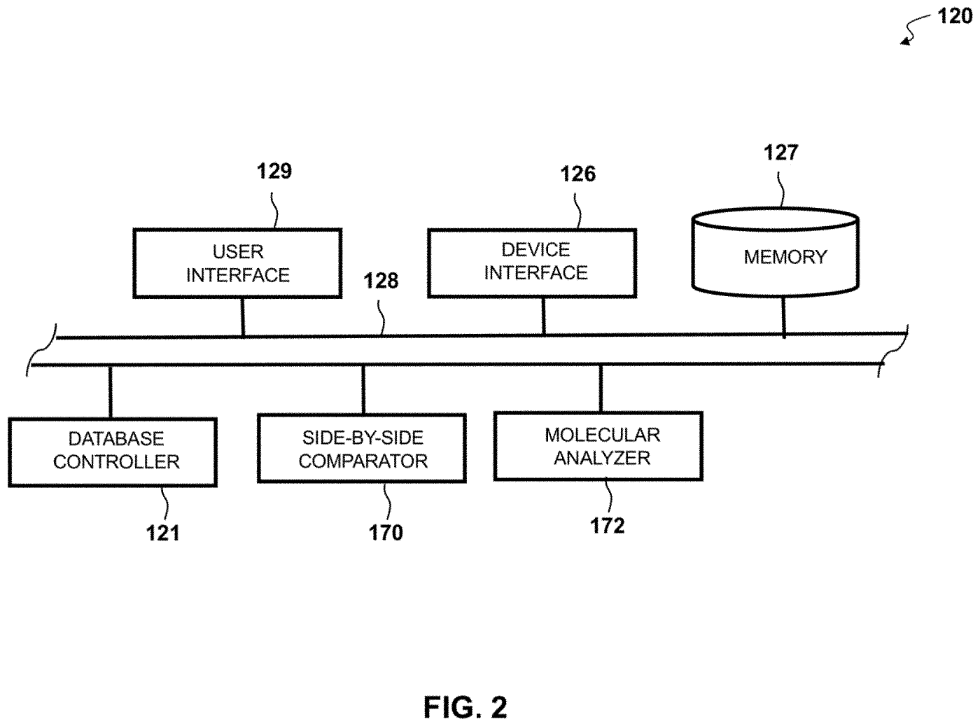

With respect to FIG. 2, components associated with or in communication with the processor, 124 are shown. A database controller 121 may be in communication with the processor 124, for example, via the data bus 128. In one embodiment, the 10 database controller 121 may receive and store data, such as data from various industries (e.g., pharmaceutical industry, chemical industry, FDA, etc.) as well as a library of different molecules from at least one database, such as a database associated with the server computer 140 in FIG. 1, and load said data into, for example, a cross-platform database program. More specifically, “Head Start Molecules” are molecules that a user may, at their discretion, add to the database, the head start molecules having different molecular structures or are molecules known to have certain desirous properties that are related to a user defined metric goal. A user may launch the targeted molecular design application (e.g., application 122) to interact with the program at the user interface 129. Each molecule in this database may then be measured with a molecular analyzer component 172 for many different molecular attributes, including all molecule metric goals and the like. In one embodiment, the molecular binding affinity may be measured with the molecular analyzer component 172. In another embodiment, the binding affinity of a molecule to a target protein is measured (determined) using third-party protein-ligand docking software, wherein a simulation of the molecular docking between the molecule (ligand) and target receptor (protein) is performed in order to predict the real-world binding energy and/or binding affinity of a chemical interaction between the two. In one embodiment, additional molecular properties may be measured using open-source software (e.g., RDKit).

The analyzed molecules and their corresponding properties may be saved in a file within an output folder chosen or selected by a user. A side-by-side comparator component 170 may receive the analyzed molecular data to perform advanced analytics on the data to provide a side-by-side comparison of properties of two or more molecules which can allow users to quickly and easily view and compare top candidate molecules. Here, user selected properties can be displayed, or a default set of properties will be displayed.

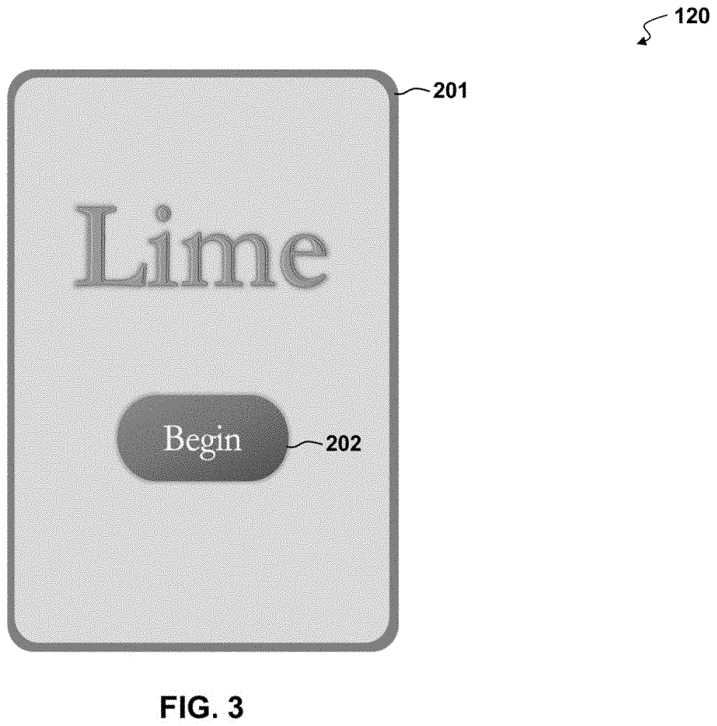

With respect to FIG. 3, the system 100 may provide for targeted molecular design allowing a user to design molecules having desired traits, and automatically providing detailed metrics for the new molecules to the user or users. In one embodiment, the side-by-side comparator component 170 may automatically present to the user organized, easy to understand, and sortable measurements of all newly generated molecules at the user interface 129, allowing the user to immediately view side-by-side comparisons of all relevant properties in new molecules.

In one embodiment, the user may be presented with a welcome screen 201 with a “begin” toggle button 202 at the computing device 120. In one embodiment, the user may be presented with the welcome screen upon launching the targeted molecular design application.

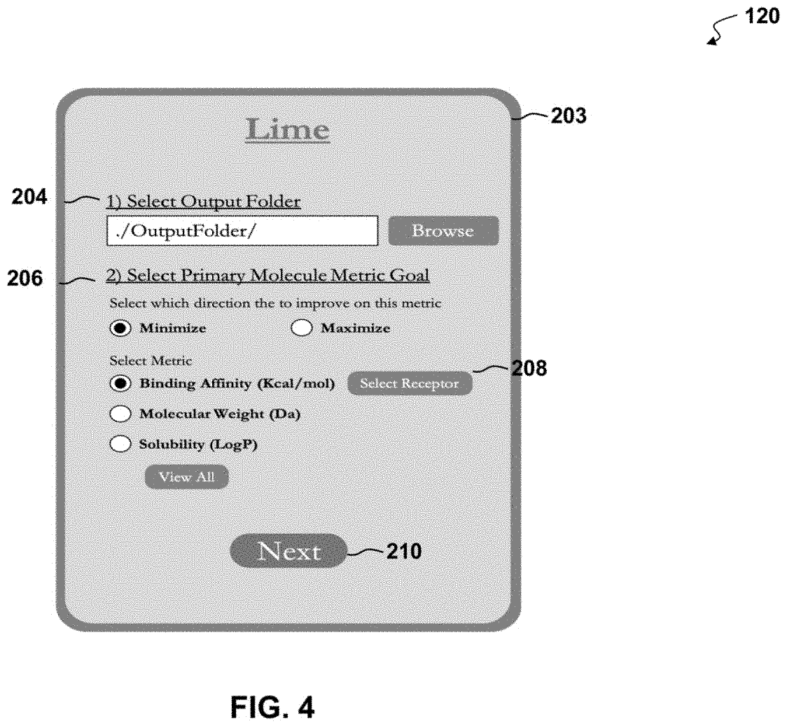

Once the user selects the “Begin” button 202, the user is taken to a settings page 203, as shown in FIG. 4. The settings page displays the required settings at the user interface that the user must provide or enter in order to run the targeted molecular design application. In one embodiment, a first setting 204 is selected by the user at the user interface 129 to choose an output folder on a computing device, such as computing device 120 where the application 122 saves all molecule information and other files it creates. For example, a “Training Smiles” folder may contain text documents of the top-scoring molecules which are selected for training at each generation. Additionally, a “Model Checkpoints” folder may save at least one Hierarchical Data Format version 5 (HDF5) file, which includes the system's LSTM training checkpoints. Finally, a “Results” Folder may include separate folders for each “Generation” of designed molecules, described in further detail below. In one embodiment, each Generation folder may contain “Docking Logs” of each generated molecule's simulated docking, i.e., binding, with the target protein, 3-D Files in Protein Data Bank (PDB) format of each molecule, 3-D Files in PDBQT format (similar to PDB format but including partial charges (‘Q’) and AutoDock 4 (AD4) atom types (‘T’)) showing the most probable docking configurations found in each docking simulation, and a “MoleculeMetrics.csv” file, which may contain all of the molecule measurements for each “Generation”. After a “Generation” is complete, the “MoleculeMetrics.csv” file may be added to the previous generation's master table (if the file is not the first generation (generation 0)), and the new, combined table may be saved as that generation's new master table in the “MasterResults” folder. In another embodiment, additional files may be included. In yet another embodiment, the system may have a user-friendly, icon-based file organization.

A second setting 206 is then selected by the user at the user interface 129 to choose a “Primary Molecule Metric Goal”. In one embodiment, the Primary Molecule Metric Goal input selected by the user may be received at the molecular analyzer component 172 indicating the most important molecular quality that the molecular analyzer component 172 may design molecules to have. For example, if a user wishes to design a cure for a specific disease, the user may select “Minimize” and “Binding Affinity” as their primary metric (as shown in FIG. 4), which tells molecular analyzer component 172 to design molecules with the lowest binding affinity, for example, lowest equilibrium disassociation constant K_{D}, with a virus' target receptor. In other words, the most important quality when designing a cure for a disease would be the drug's ability to combat the disease, and this would take precedence over other molecular attributes of the drug. When designing molecules for a different purpose, the user may similarly select any desired metric as their primary molecule metric goal. In another embodiment, the binding affinity may be calculated with a third-party program. In another embodiment, the binding affinity may be calculated with the molecular analyzer component 172.

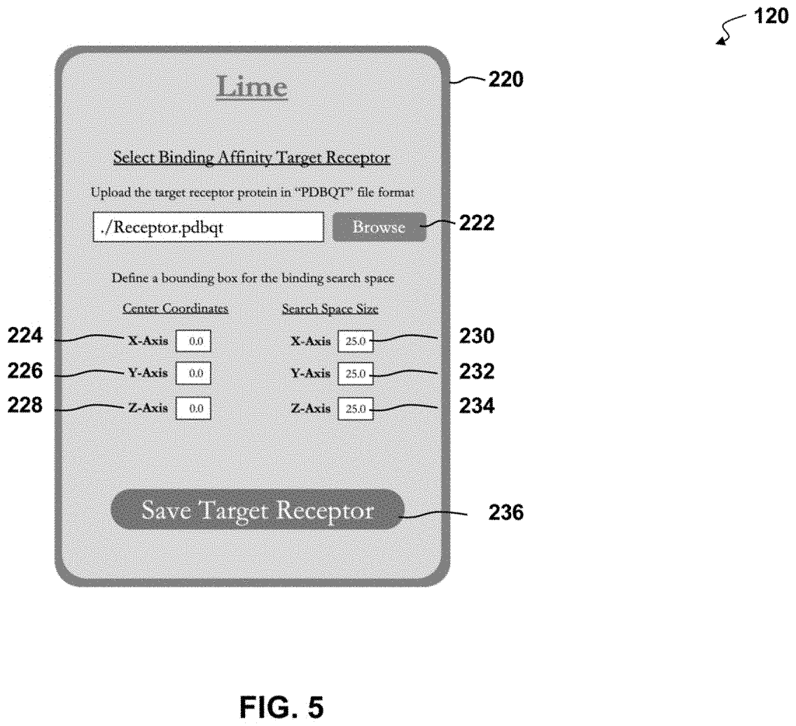

In one embodiment, when the user selects binding affinity as a primary or secondary molecule metric goal, the user must select the “Select Receptor” button 208 which opens a Receptor Selection page 220 shown in FIG. 5. At the Receptor Selection page 220, the user may upload, with a Browse button 222, a protein structure file of the receptor that they wish to target as a “.PDBQT” file, a common type of file used in drug discovery. For example, if a user wanted to design a drug to combat COVID-19, the user may select to upload a “.PDBQT” file of the Covid-19 Spike Protein, which is used by the virus to enter human cells. This protein structure is used by the molecular analyzer component 172 to predict all of the generated molecules' binding affinity to the Spike protein. As such, the molecular analyzer component 172 measures how well each generated molecule would be able to prevent COVID-19 from entering human cells by binding to the target receptor of the spike protein. In turn, the system 100 will learn, based on the scoring of the molecules, the molecular qualities that help drugs inhibit the virus and design original, new drugs with more of these qualities.

Once the user has uploaded the receptor file, i.e., the binding target, the user needs to define a bounding box, which dictates which part of the receptor will be analyzed when determining binding affinity of the different molecules to the receptor on the virus. In one embodiment, the user enters numerical values for center coordinates to center on the receptor. The center coordinates may be x, y, and z coordinate values entered at an X-axis 224, Y-axis 226, and Z-axis 228 coordinate boxes, respectively. In one embodiment, the boxes 224, 226, 228 may have a default value of 0.0. In one embodiment, the user enters numerical values for the three-dimensional search space size of the receptor within which the ability of the molecule(s) to bind thereto will be evaluated. The search space size may be x, y, and z coordinate values entered at an X-axis 230, Y-axis 232, and Z-axis 234 coordinate boxes, respectively.

In one embodiment, the boxes 230, 232, 234 may have a default value of 25.0. Angstrom units (angstroms). Once the user has entered all of the receptor information, the user may press a “Save Target Receptor” button 236 which will save the receptor information and return the user to the previous settings screen 203.

In one embodiment, once the Required Settings Screen 203 is complete, the user may click a “Next” button 210 at the Required Settings Screen 203 to move to an Optional Settings Screen 240. The first optional setting allows the user to add secondary molecule metric goals. In one embodiment, while the user may only add one primary molecule metric goal, the user may add as many secondary molecule metric goals as the user desires. In one embodiment, while these secondary molecule metric goals are given lower priority than the primary goal, they are still factored into the design of new molecules by the molecular analyzer component 172.

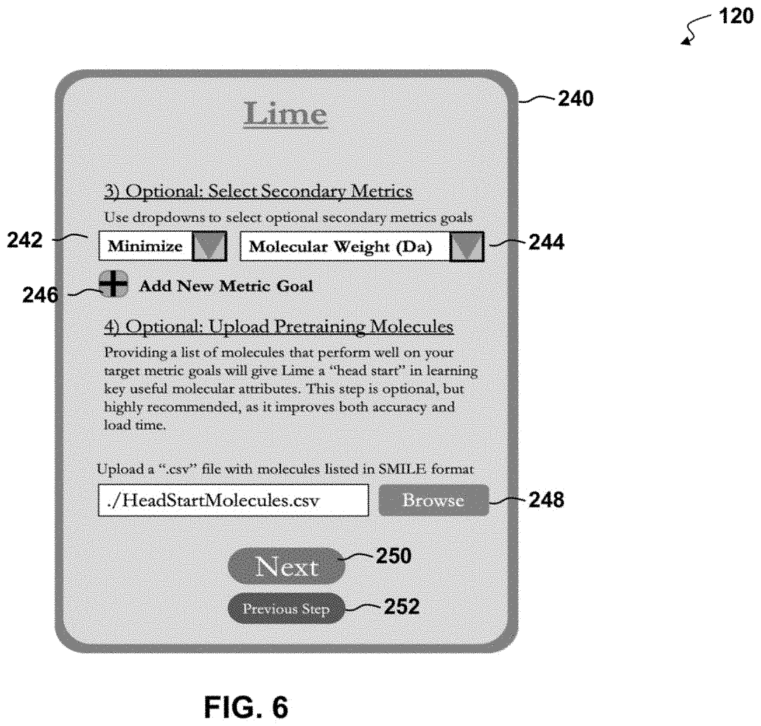

For example, and with respect to the COVID-19 virus, the most important molecular quality needed for a candidate cure would be the molecule's ability to prevent the virus from entering cells, and thus the binding affinity is the overall gating metric for potential applicability of a new molecule as a potential treatment for the Covid-19 infection. However, an ideal drug must also have a low molecular weight, along with several other key molecular attributes, in order for the drug to be absorbed by the body. Therefore, the user could enter these important drug attributes at a first button 242 and a second, related button 244, as shown in FIG. 6, and all of the other molecular attributes with an Add New Metric Goal 246. Thus, multiple secondary molecular attributes can be added and evaluated along with the primary evaluation attribute.

Additionally, the user may provide additional “pretraining” molecules hereafter, “head start” molecules, to enhance the system's 100 learning. For example, while COVID-19 is a novel virus with no known cures, the virus shares similarities with several other viruses that have been well researched, such as HIV and SARS. In one embodiment, the user may provide the molecular analyzer component 172 with drugs that are already known to combat HIV or SARS. This may provide the software a head start in learning the key attributes that help inhibit similar diseases to COVID-19, which, in turn, allows the software to learn which of the attributes are also helpful for inhibiting COVID-19 and apply the attributes to the design of the new drug. In one embodiment, if the user chooses to provide such molecules, the user may upload a file, such as a “.csv” file by selecting a Browse button 248 in the molecular attributes 240 screen interface. In one embodiment, the file may contain a list of these molecules in SMILE format, and it will improve both the learning speed and performance of the software. Once the user has completed the optional settings, the user may press a “Next” button 250 in the molecular attributes 240 screen interface to be taken to a Summary Screen 260 displayed at the user interface 129. In one embodiment, the Summary Screen 260 provides a list 262 of all the settings chosen by the user for confirmation. The user may go back to change their settings using the “Previous Step” button 252, or if the user does not wish to make changes, they may click the “Start” button 250 to begin the molecule design process.

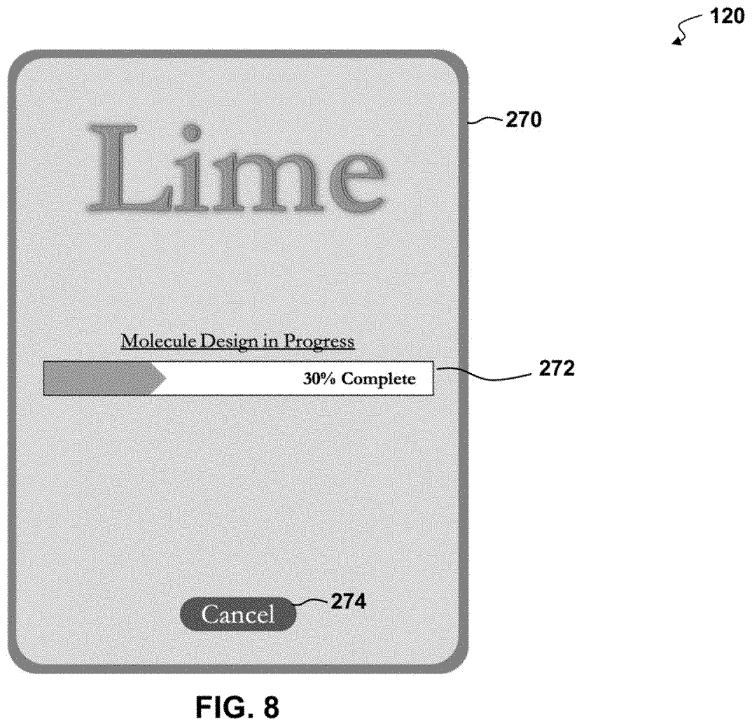

Upon clicking the Start button 252, the targeted molecule design process may begin automatically, and the user is directed to a Progress Bar Screen 270, as shown in FIG. 8. In one embodiment, the Progress Bar Screen 270 may include a progress bar 272, where the user may view the percentage of the process completed by the targeted molecular design application 122. The user may cancel the process at any time by selecting a cancel button 274.

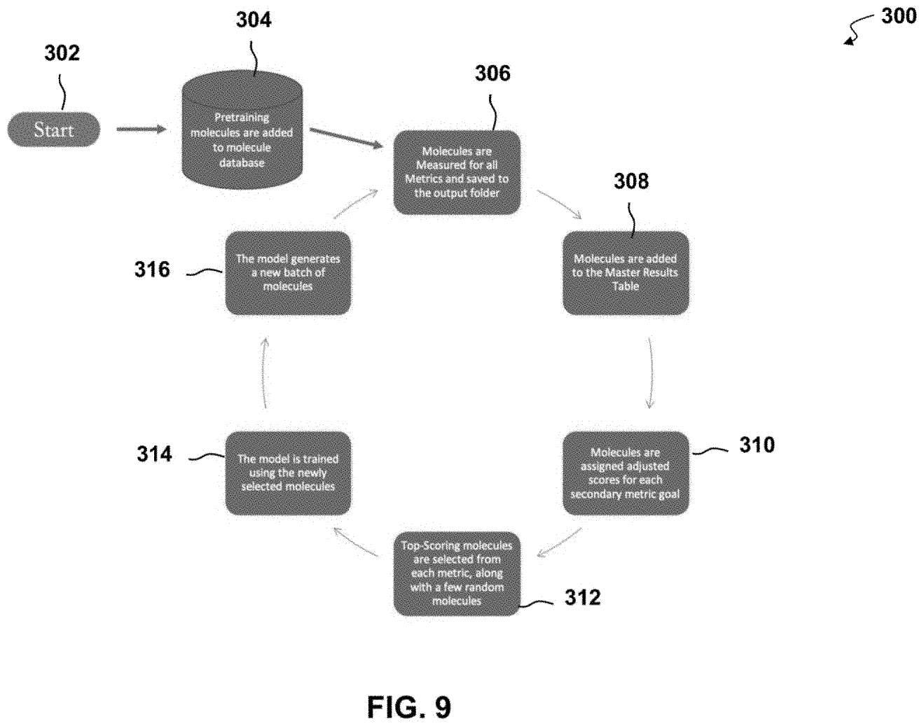

With respect to FIG. 9, a flow chart 300 depicts an iterative process for targeted molecular design. At a first step 302, the process is initiated. At a step 304, the “Head Start Molecules” may be added to the database, the head start molecules containing different molecules. In one embodiment, the “Head Start Molecules” may be uploaded in an Optional Settings Page (see FIG. 6) prior to starting the molecular design process. The Head Start Molecules may be uploaded by entering the full file-path to the user's document containing the “Head-Start Molecules”, or by clicking a “Browse” Button (see Browse Button 248 of FIG. 6). The molecules will then be added to the standard list of molecules and scored along with the standard list of molecules during the first batch of measurements and top molecules may be selected from the full, joined list. The standard list of molecules is a set of molecules stored in the system memory and is used as the initial molecules for evaluation by the molecular design system. After analyzing and scoring the head start molecules against the desired metrics, the molecular design system uses the highest scoring molecules as measured against the user selected target metrics, to iteratively design additional molecules having higher scores against the target metrics. By adding the user selected head start molecules to the standard list of molecules previously stored in the molecule design system, the system receives a users' input regarding what preexisting molecules that the user believes will be the closest to the ultimately designed molecule, and thus provides a “head start” to the system molecule design process.

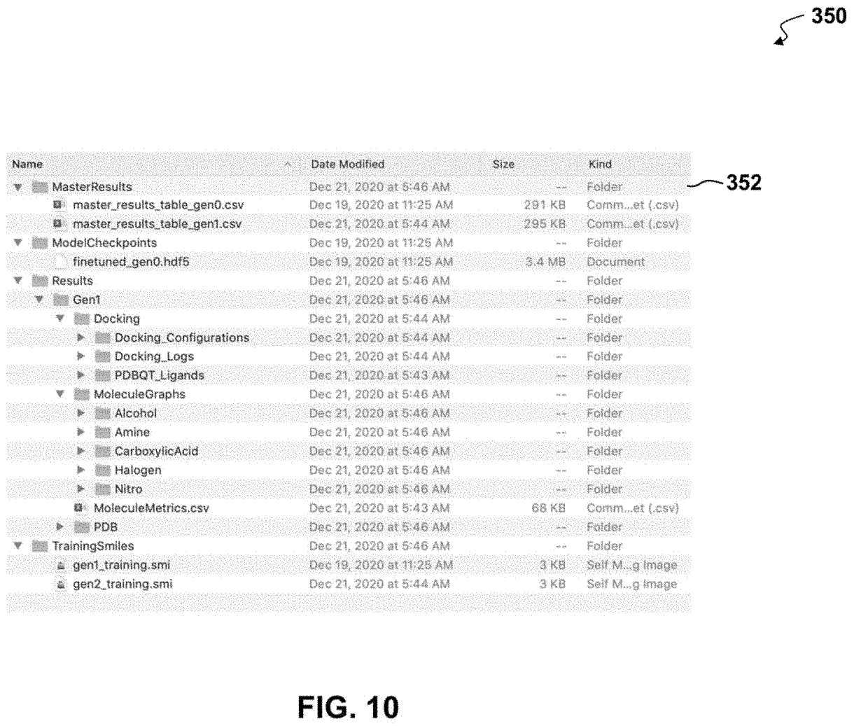

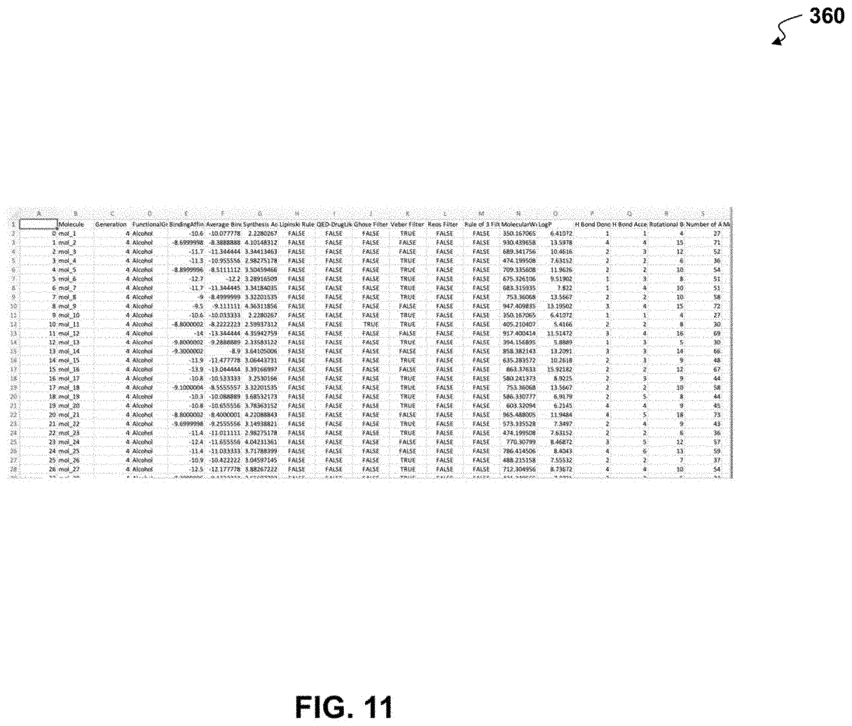

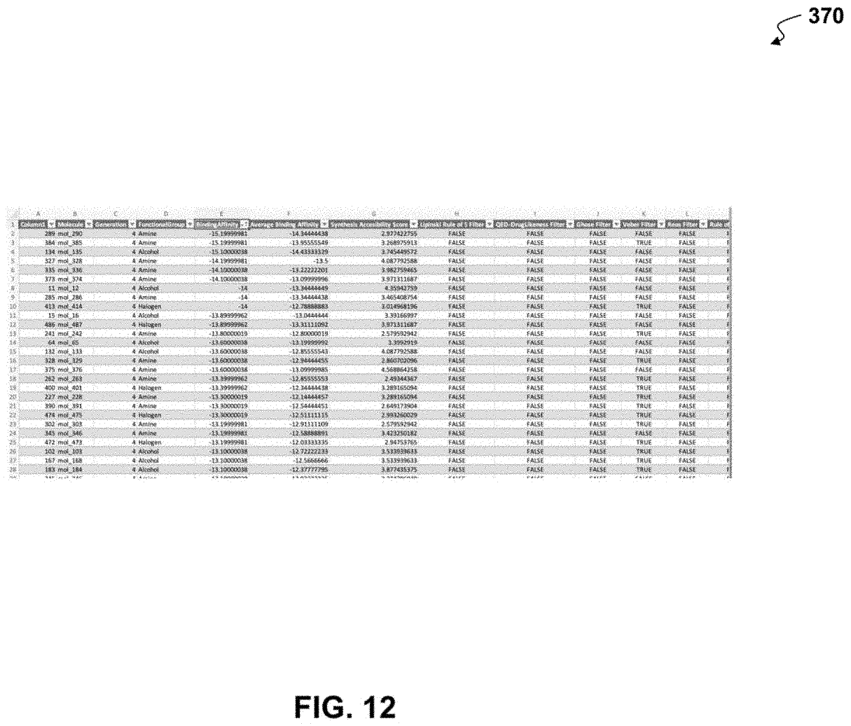

Each molecule in the standard molecule database, along with any “Head Start Molecules” added by the user, is then measured for many different molecular attributes at a step 306, including all molecule metric goals and more. The metrics are then saved to an output folder. In one embodiment, all of the molecules and their corresponding properties are saved within an output folder 350, shown in FIG. 10. In one embodiment, the output folder is selected by the user on the Required Setting Screen 203. A file, such as a “.csv” file 360 shown in FIG. 11, with all of the molecules and their corresponding properties is saved to the output folder. The file 360 may be easily converted into a sortable, filterable table 370, such as an Excel file shown in FIG. 12. The table 370 may allow users to quickly and easily view and compare top candidate molecules.

Along with the “.csv” file 360 shown within a MasterResults folder 352, the folder contains additional subfolders: “MoleculeGraphs”, “Docking”, and “PDB”.

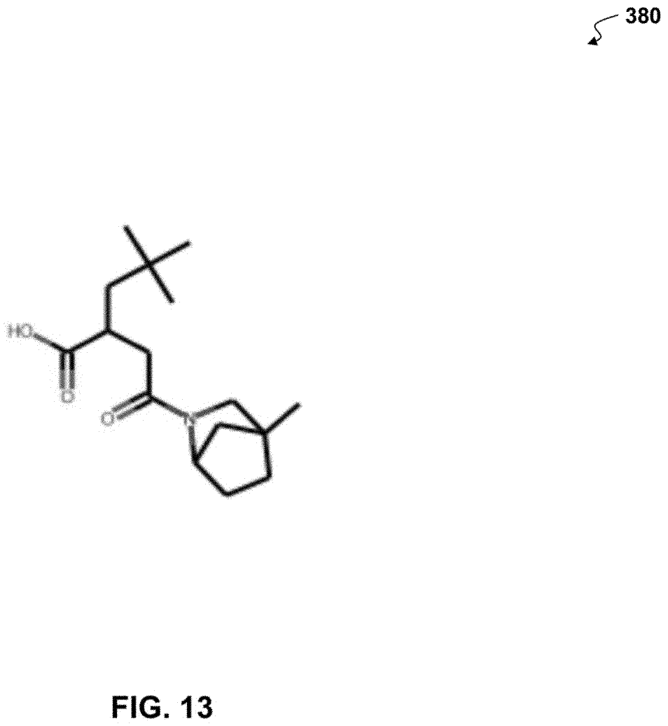

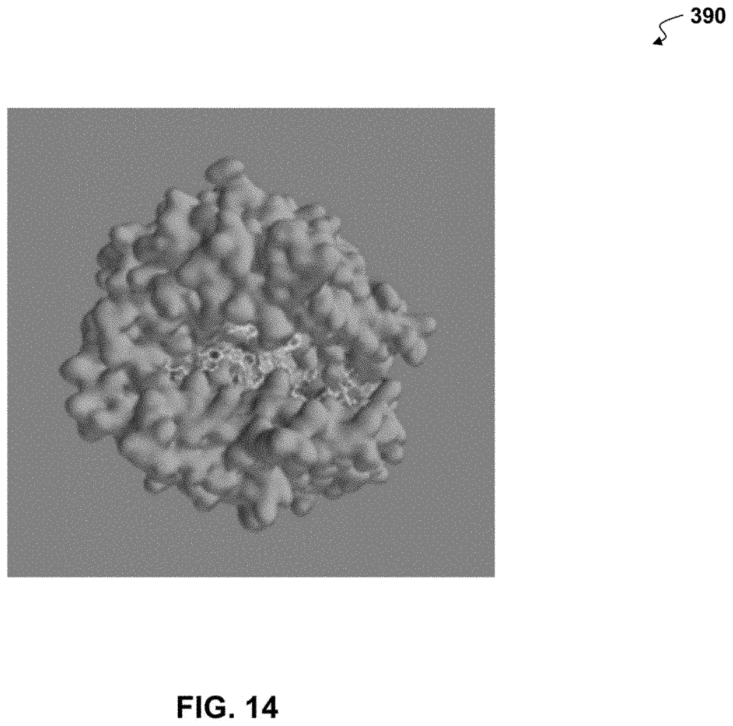

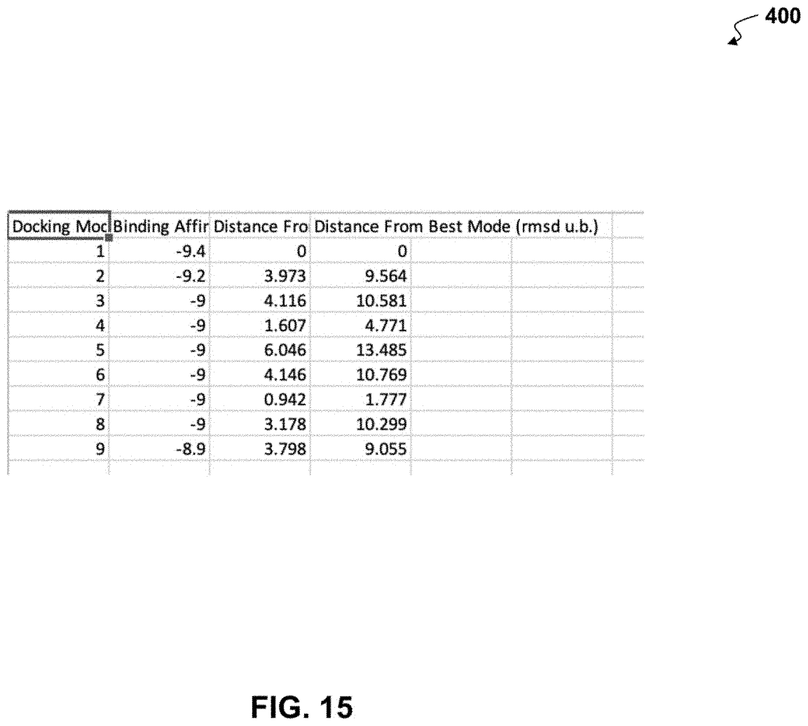

The “MoleculeGraphs” folder may contain molecular graph images, such as molecular image 380 of FIG. 13 and molecular image 390 of FIG. 14. The molecular images 380, 390 provide 2-D representations of each molecule, conveniently stored into subfolders organized by molecular functional group. In one embodiment, and with respect to FIG. 15, the “Docking” folder contains a detailed log 400 from the molecular analyzer component 172 or the third-party protein-ligand docking software for each molecule along with three dimensional (3-D) “. PDBQT” files containing the target receptor, and the highest probability positions in which the ligand, i.e., the molecule or a portion thereof, would bind to it. In another embodiment, the binding affinity may be calculated with the molecular analyzer component 172.

At a step 308, molecules and their corresponding measurements may be added to a Master Results table. More specifically, once all of the molecule metrics have been saved, a copy of the table in “MoleculeMetrics.csv” may be saved as the initial master-table under the name “master_results_table_gen0.csv” (or genX, depending on the generation). More specifically, the original molecules of the standard list of molecules dataset along with “Head Start Molecules” provided by the user are designated as gen0, and the first batch of original molecules generated by the system are gen1. The generations continue to increment by 1 with each new batch of molecules. Once the newly generated batch of molecules are measured, the molecules and their corresponding measurements are combined with the previous generations' “master_reults_table_gen0.csv” and the combined table may then be saved as “master_reults_table_gen1.csv”, with each generation creating a new, Master Results table with that generations molecules and measurements along with all previously measured molecules.

At the outset, the “MasterResults” subfolder 352 may be saved within the base output folder 350, and the iterative molecule design process begins. First, using the master results table, the molecules with the highest scores on the primary molecule metric goal are selected. For example, where the user selects the number of molecules having the highest score on the primary metric, that number of molecules, hiving the highest score on that metric, will be chosen. Then, at a step 310, all molecules are given an adjusted score for each secondary molecule metric goal based on a combination of their scores on both the primary and secondary molecule metric goal. At a step 312, the molecules with the highest adjusted scores are then selected for each secondary metric goal. For example, the number of molecules the user has selected for the secondary goals will be selected, based on the highest score on the secondary metric(s), an adjusted score. A baseline long short-term memory (LSTM) network (or other form of sequence-generating neural network) model trained to generate a wide variety of different molecules may then generate at least one random molecule to introduce random mutation, and then the at least one random molecule(s) is combined with the previously selected molecules and saved in SMILE format in the “TrainingSmiles” folder found in the base output folder.

At a step 314, a copy of the baseline LSTM model mentioned above is then trained using these molecules newly added to the TrainingSmiles folder, where the system/program learns to design new molecules combining substructures and molecule properties of all the top-scoring molecules from the Training Smiles folder.

The newly trained LSTM model is then used to generate a batch of new molecules, at a step 316. The new molecules are then scored using the same process as was performed on the molecules in the original database, but when creating copying the results to the new master table, the table is first combined with the previous masters table, and then saved as the new master table of the next generation.

This process proceeds iteratively back to step 306 and is repeated over and over, gradually training the LSTM model to generate new molecules using the previously generated molecules with continuously improved results across all desired molecule metric goals, and saving each new generation of all molecule metrics/files to the output folder after each new batch of new molecules.

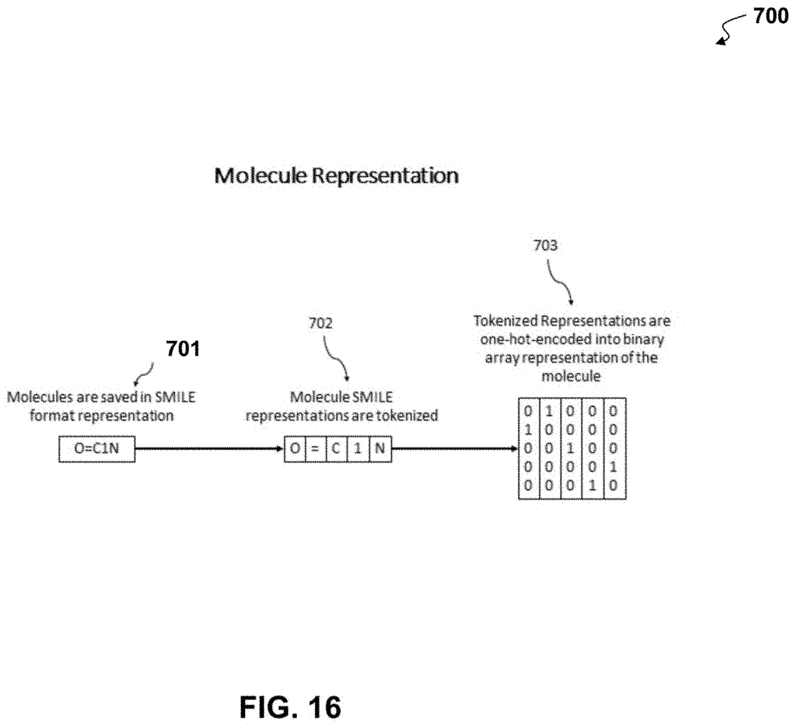

With respect to FIG. 16, a flow diagram 700 of a molecular representation is illustrated. At a step 701, molecules may be stored in SMILE format, as described above. At a step 702, the molecules in smile format may be tokenized to split the SMILES format representation thereof into a vector of individual pieces of the SMILES format representation of the molecule. Tokenizing the SMILES format representation of the molecule breaks the SMILES text into individual linguistic units. More specifically, the SMILE format representation molecules may be split into each individual character, so the Molecular Generator network may be able to generate the SMILE string by selecting one character (e.g., letter, number, symbol, or “end-sequence” token) at each timestep of generation. At a step 703, each individual piece or element of the vector representation of the SMILES format of the molecule may be one-hot-encoded into a binary representation, resulting in a binary array representation of each molecule. This binary array representation is then used for the baseline of the long short-term memory (LSTM) Molecule Generator network (or other form of sequence-generating neural network).

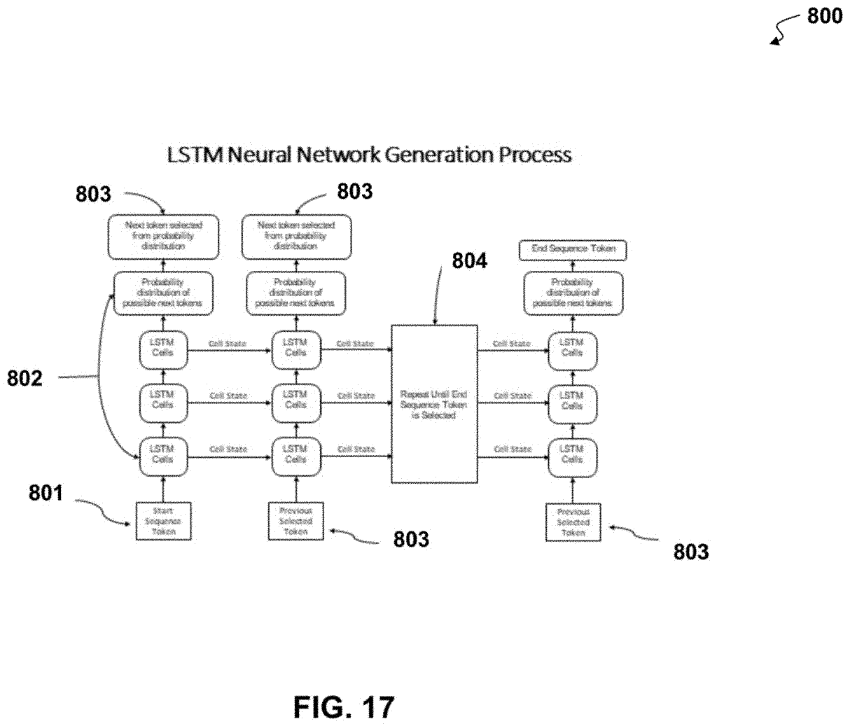

With respect to FIG. 17, a flow diagram 800 of the LSTM network is shown. At a step 801, a start token may be given to the LSTM Molecular Generator Network as input to begin sequence generation. At a step 802, the LSTM Molecular Generator Network outputs a probability distribution predicting the likelihood of each possible token being the next token in a molecular sequence. At a step 803, a token is sampled from the probability distribution (with temperature to increase variation), and used as the next input token for the LSTM Molecular Generator Neural Network. At a step 804, process is repeated until the “End Sequence Token” is selected. The full sequence of predicted tokens may be converted into a molecule represented in SMILE format.

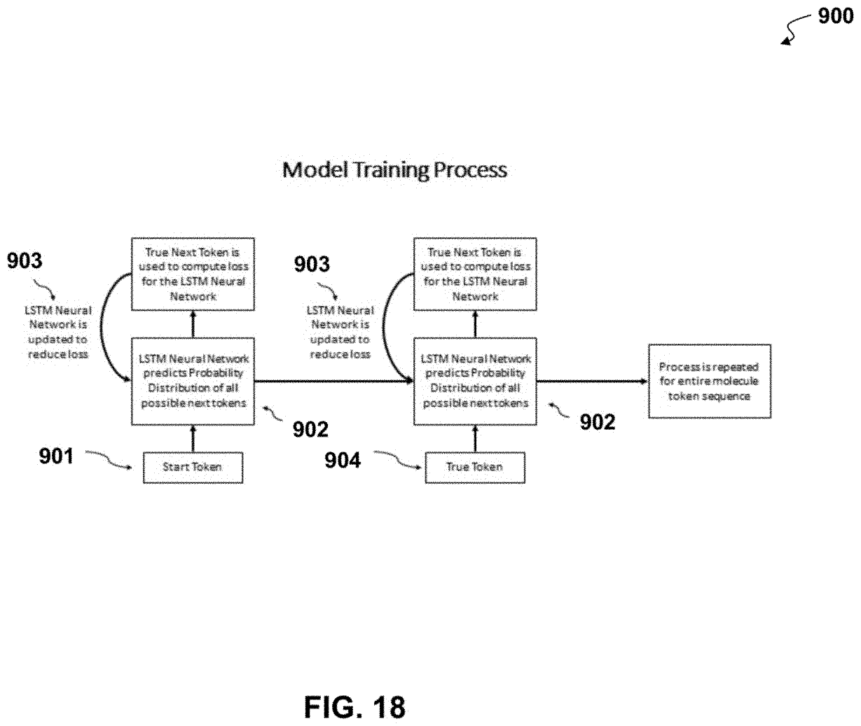

With respect to FIG. 18, a flow diagram 900 of a model training process is shown. At a step 901, tokenized molecule sequences, such as Tokenized Molecule sequences described in FIG. 16 are fed to the LSTM Molecular Generator Network as input. At each timestep, the LSTM Molecular Generator Network outputs a probability distribution of the likelihood of each possible token being the next token in the molecule sequence, at a step 902. At a step 903, the LSTM Molecular Generator Network maybe updated to assign a higher probability to the true next token found in the training molecule, causing the LSTM Molecular Generator Network to generate molecules more similar to the training molecules. At a step 904, the true next token of the training molecule may be used as the input of the LSTM Molecular Generator Network for the next timestep in the sequence, and this process may be repeated until all molecules are complete.

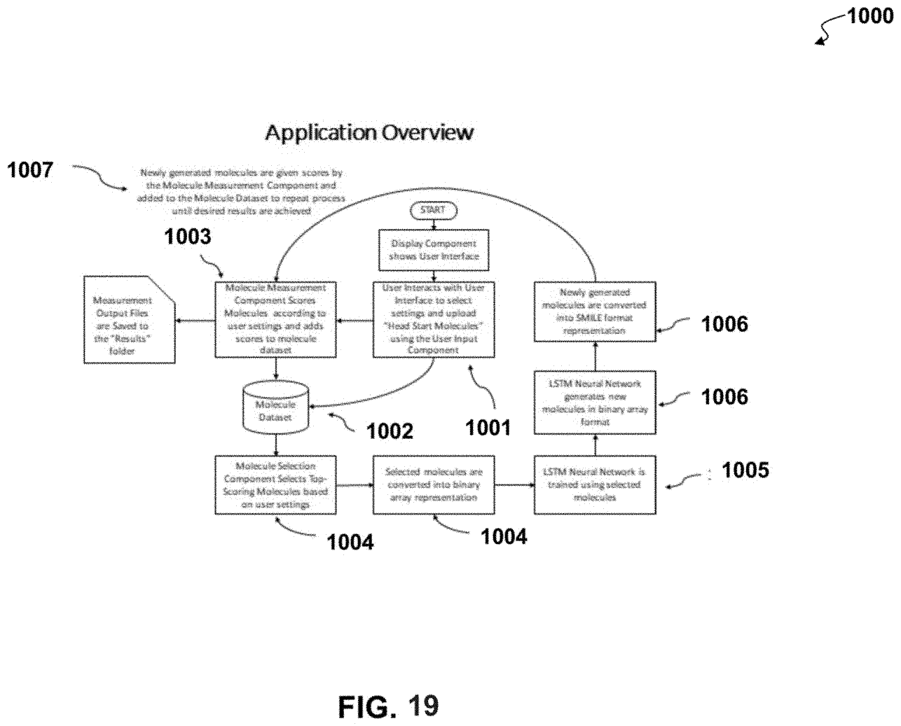

With respect to FIG. 19, a flow diagram 1000 of an overview of a system for automated targeted molecular design is shown. At a step 1001, a user uses the computer keyboard and mouse to input user settings, upload “Head Start Molecules” in SMILE format, and begin the molecule design process. At a step 1002, the “Head Start Molecules” are added to a pre-existing dataset of molecule in SMILE format stored in a memory component, i.e., the standard dataset. At a step 1003, the full molecule dataset is given to the molecule measurement component along with the user settings to assign each molecule scores for all metrics. All scores are added to the molecule dataset and all measurement output files are saved to a “Results” folder. At a step 1004, the molecule dataset is given to a molecule selection component, and the top-scoring molecules are selected, converted into binary array representations and given to a LSTM Molecular Generator Network. The LSTM Molecular Generator Network is trained with the selected molecules. At a step 1005, the LSTM Molecular Generator Network generates new molecules more similar to the selected molecules, which are converted to SMILE format and given to the molecule measurement component. At a step 1006, the molecule measurement component scores the new molecules and adds them, along with all of their scores to the molecule dataset. At a step 1007, steps 1003 through 1007 are repeated until desired results are achieved.

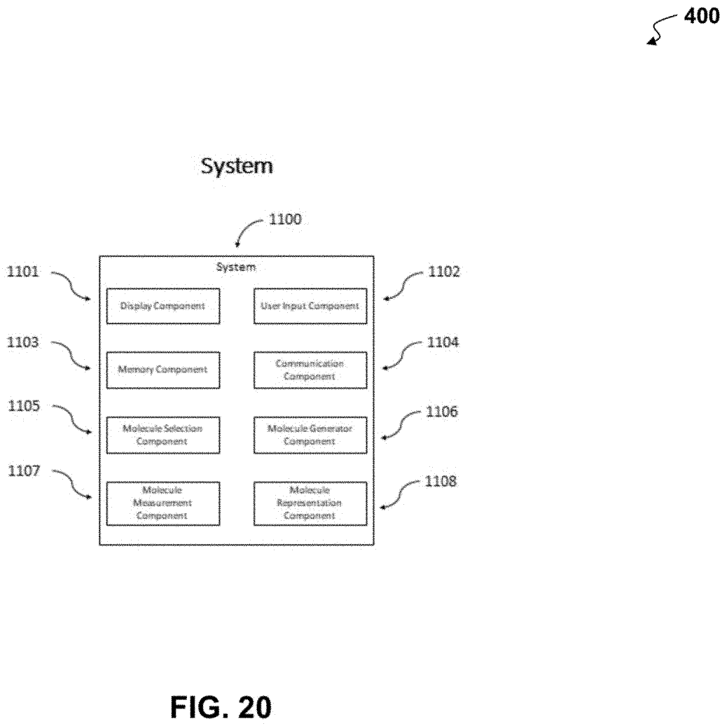

With respect to FIG. 20, a block diagram of the system 1100 for automated targeted molecular design is shown. The system (1100) may include a Display Component (1101), a User Input Component (1102), a Memory Component (1103), a Communication Component (1104), a Molecule Selection Component (1105), a Molecule Generator Component (1106), a Molecule Measurement Component (1107), and a Molecule Representation Component (1108). In one embodiment, the Display Component (1101) displays the User Interface on the System (1100), which the user may interact with using the User Input Component (1102). In one embodiment, the User Input Component (1102) may consist of a keyboard and/or mouse, a touchscreen in another embodiment, or other input devices in other embodiments. The Memory Component (1103) may contain a dataset of molecules represented in SMILE format representation, binary array representation or molecule representations. The Memory Component (1103) may additionally include scores, measurements, logs, or other data corresponding to the molecules within the dataset.

The Communication Component (1104) may be configured to establish a connection between the System (1100) and any number of external molecule databases in order to send and/or retrieve additional molecule data for the Memory Component (1103). The Molecule Selection Component (1105) may be configured to select top-scoring molecules from the Memory Component (1103) based upon the user settings provided by the User Input Component (1102) and the molecule measurements and/or scores created by the Molecule Measurement Component (1107).

The Molecule Generator Component (1106) may consist of one or many LSTM Molecular Generator Networks used to generate new molecules in a binary array representation. The Molecule Measurement Component (1107) may be configured to assign measurements and/or scores to large lists of molecules, for any number of molecular attributes defined by the user settings received by the User Input Component (1102). The Molecule Representation Component (1108) may be configured to convert the representations of molecules between different molecular representation including but not limited to SMILE format representation, binary array representation, 3-D structural graph representation, and any other molecular representation format needed by other components within the System (1100).

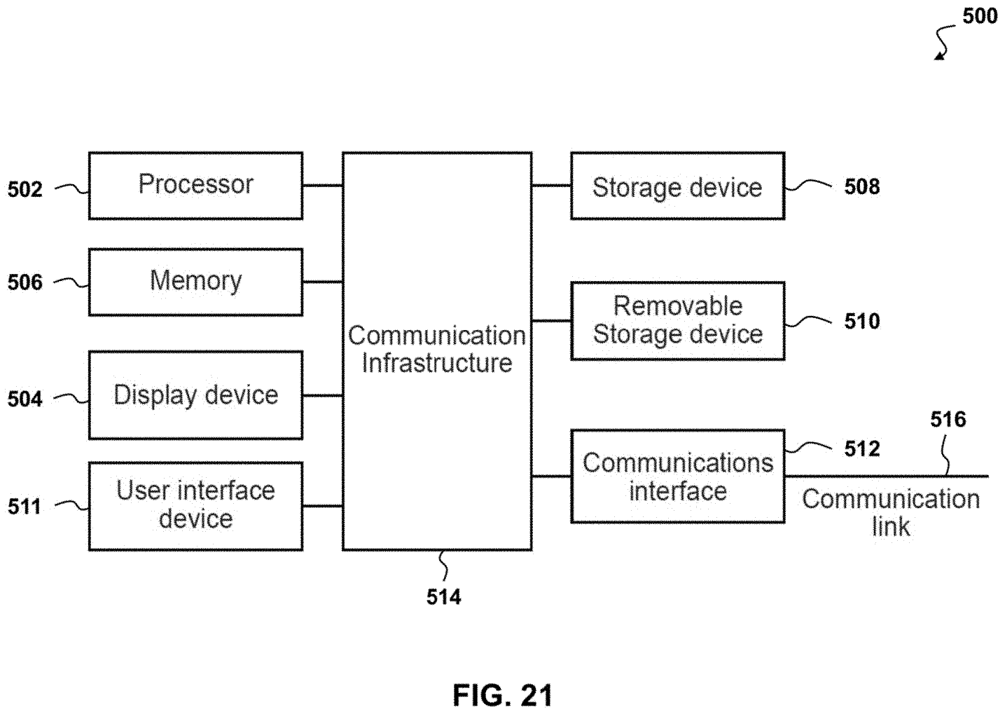

FIG. 21 is a high-level block diagram 500 showing a computing system comprising a computer system useful for implementing an embodiment of the system and process, disclosed herein. Embodiments of the system may be implemented in different computing environments. The computer system includes one or more processors 502, and can further include an electronic display device 504 (e.g., for displaying graphics, text, and other data), a main memory 506 (e.g., random access memory (RAM)), storage device 508, a removable storage device 510 (e.g., removable storage drive, Graphics Processing Unit (GPU), a removable memory module, a magnetic tape drive, an optical disk drive, a computer readable medium having stored therein computer software and/or data), user interface device 511 (e.g., keyboard, touch screen, keypad, pointing device), and a communication interface 512 (e.g., modem, a network interface (such as an Ethernet card), a communications port, or a PCMCIA slot and card). The communication interface 512 allows software and data to be transferred between the computer system and external devices. The system further includes a communications infrastructure 514 (e.g., a communications bus, crossover bar, or network) to which the aforementioned devices/modules are connected as shown.

Information transferred via communications interface 514 may be in the form of signals such as electronic, electromagnetic, optical, or other signals capable of being received by communications interface 514, via a communication link 516 that carries signals and may be implemented using wire or cable, fiber optics, a phone line, a cellular/mobile phone link, an radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer-implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface 512. Such computer programs, when executed, enable the computer system to perform the features of the embodiments as discussed herein. In particular, the computer programs, when executed, enable the processor and/or multicore processor to perform the features of the computer system. Such computer programs represent controllers of the computer system.

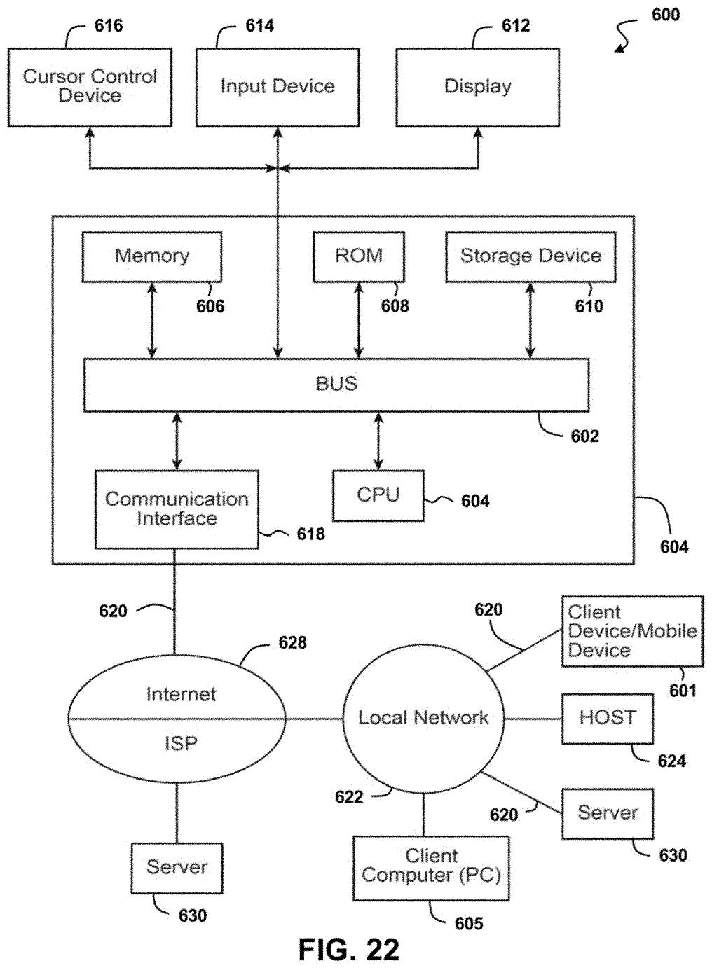

FIG. 22 shows a block diagram of an example system 600 in which an embodiment may be implemented. The system 600 includes one or more client devices 601 such as consumer electronics devices, connected to one or more server computing systems 630. A server 630 includes a bus 602 or other communication mechanism for communicating information, and a processor (CPU and/or GPU) 604 coupled with the bus 602 for processing information. The server 630 also includes a main memory 606, such as a random access memory (RAM) or other dynamic storage device, coupled to the bus 602 for storing information and instructions to be executed by the processor 604. The main memory 606 also may be used for storing temporary variables or other intermediate information during execution or instructions to be executed by the processor 604. The server computer system 630 further includes a read only memory (ROM) 608 or other static storage device coupled to the bus 602 for storing static information and instructions for the processor 604. A storage device 610, such as a magnetic disk or optical disk, is provided and coupled to the bus 602 for storing information and instructions. The bus 602 may contain, for example, thirty-two address lines for addressing video memory or main memory 606. The bus 602 can also include, for example, a 32-bit data bus for transferring data between and among the components, such as the CPU 604, the main memory 606, video memory and the storage 610. Alternatively, multiplex data/address lines may be used instead of separate data and address lines.

The server 630 may be coupled via the bus 602 to a display 612 for displaying information to a computer user. An input device 614, including alphanumeric and other keys, is coupled to the bus 602 for communicating information and command selections to the processor 604. Another type of user input device comprises cursor control 616, such as a mouse, a trackball, or cursor direction keys for communicating direction information and command selections to the processor 604 and for controlling cursor movement on the display 612.

According to one embodiment, the functions hereof are performed by the processor 604 executing one or more sequences of one or more instructions contained in the main memory 606. Such instructions may be read into the main memory 606 from another computer-readable medium, such as the storage device 610. Execution of the sequences of instructions contained in the main memory 606 causes the processor 604 to perform the process steps described herein. One or more processors in a multiprocessing arrangement may also be employed to execute the sequences of instructions contained in the main memory 606. In alternative embodiments, hardwired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor 604 for execution.

Such a medium may take many forms, including but not limited to, non-volatile media, volatile media, and transmission media. Non-volatile media includes, for example, optical or magnetic disks, such as the storage device 610. Volatile media includes dynamic memory, such as the main memory 606. Transmission media includes coaxial cables, copper wire and fiber optics, including the wires that comprise the bus 602. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CDROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor 604 for execution. For example, the instructions may initially be carried on a magnetic disk of a remote computer. The remote computer can load the instructions into its dynamic memory and send the instructions over a telephone line using a modem. A modem local to the server 630 can receive the data on the telephone line and use an infrared transmitter to convert the data to an infrared signal. An infrared detector coupled to the bus 602 can receive the data carried in the infrared signal and place the data on the bus 602. The bus 602 carries the data to the main memory 606, from which the processor 604 retrieves and executes the instructions. The instructions received from the main memory 606 may optionally be stored on the storage device 610 either before or after execution by the processor 604.

The server 630 also includes a communication interface 618 coupled to the bus 602. The communication interface 618 provides a two-way data communication coupling to a network link 620 that is connected to the world wide packet data communication network now commonly referred to as the Internet 628. The Internet 628 uses electrical, electromagnetic or optical signals that carry digital data streams.

The signals through the various networks and the signals on the network link 620 and through the communication interface 618, which carry the digital data to and from the server 630, are exemplary forms or carrier waves transporting the information.

In another embodiment of the server 630, interface 618 is connected to a network 622 via a communication link 620. For example, the communication interface 618 may be an integrated services digital network (ISDN) card or a modem to provide a data communication connection to a corresponding type of telephone line, which can comprise part of the network link 620. As another example, the communication interface 618 may be a local area network (LAN) card to provide a data communication connection to a compatible LAN. Wireless links may also be implemented. In any such implementation, the communication interface 618 sends and receives electrical electromagnetic or optical signals that carry digital data streams representing various types of information.

The network link 620 typically provides data communication through one or more networks to other data devices. For example, the network link 620 may provide a connection through the local network 622 to a host computer 624 or to data equipment operated by an Internet Service Provider (ISP). The ISP in turn provides data communication services through the Internet 628. The local network 622 and the Internet 628 both use electrical, electromagnetic or optical signals that carry digital data streams. The signals through the various networks and the signals on the network link 620 and through the communication interface 618, which carry the digital data to and from the server 630, are exemplary forms or carrier waves transporting the information.

The server 630 can send/receive messages and data, including e-mail, program code, through the network, the network link 620 and the communication interface 618. Further, the communication interface 618 can comprise a USB/Tuner and the network link 620 may be an antenna or cable for connecting the server 630 to a cable provider, satellite provider or other terrestrial transmission system for receiving messages, data and program code from another source.

The example versions of the embodiments described herein may be implemented as logical operations in a distributed processing system such as the system 600 including the servers 630. The logical operations of the embodiments may be implemented as a sequence of steps executing in the server 630, and as interconnected machine modules within the system 600. The implementation is a matter of choice and can depend on performance of the system 600 implementing the embodiments. As such, the logical operations constituting said example versions of the embodiments are referred to for e.g., as operations, steps or modules.

Similar to a server 630 described above, a client device 601 can include a processor, memory, storage device, display, input device and communication interface (e.g., e-mail interface) for connecting the client device to the Internet 628, the ISP, or LAN 622, for communication with the servers 630.

The system 600 can further include computers (e.g., personal computers, computing nodes) 605 operating in the same manner as client devices 601, where a user can utilize one or more computers 605 to manage data in the server 630.

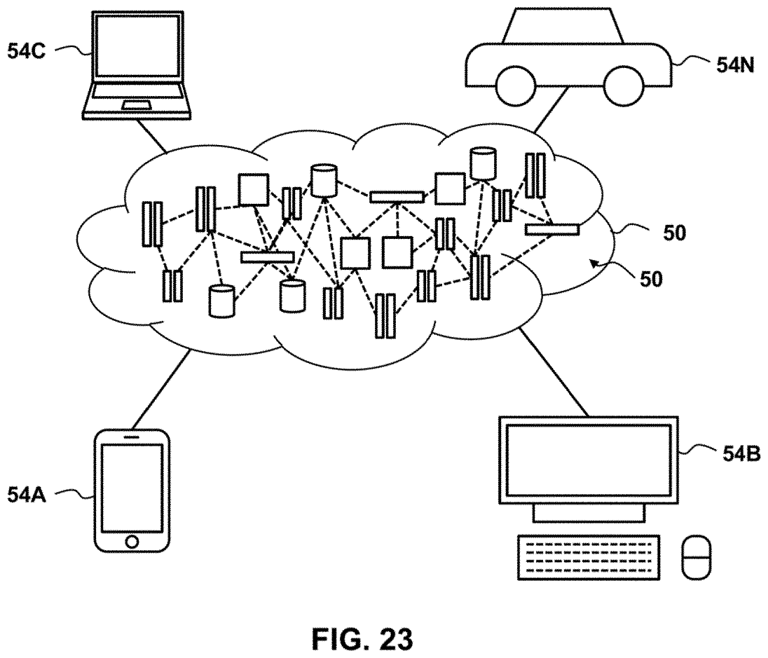

Referring now to FIG. 23, illustrative cloud computing environment 50 is depicted. As shown, cloud computing environment 50 comprises one or more cloud computing nodes 10 with which local computing devices used by cloud consumers, such as, for example, personal digital assistant (PDA), smartphone, smart watch, set top box, video game system, tablet, mobile computing device, or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 23 are intended to be illustrative only and that computing nodes 10 and cloud computing environment 50 can communicate with any type of computerized device over any type of network and/or network addressable connection (e.g., using a web browser).

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention.

Further, it is intended that the scope of the present invention is herein disclosed by way of examples and should not be limited by the particular disclosed embodiments described above.

What is claimed is:

1. A method of designing molecules having one or more desired properties, comprising:
providing a plurality of known molecules having known properties as a first dataset;
based on the properties of the known molecules, creating a plurality of new molecules having a structure different than that of at least one of the known molecules as a second data set;
evaluating the properties of the second dataset of molecules with respect to the desired properties to provide a score;
selecting a plurality of molecules from the second data set based on the score thereof to provide a nth scored dataset;
based on the properties of the second molecules, creating a plurality of new molecules in the nth data set;
selecting a plurality of molecules from the nth data set based on the score thereof to provide a nth+1 scored dataset; and
repeating the acts of creating a plurality of new molecules based on the nth+1 data set to create the nth+2 data set;
selecting a plurality of molecules from the nth+2 data set based on the score thereof to provide a nth+3 scored dataset.

2. The method of claim 1, further comprising displaying, for each designed molecule, the score thereof with respect to the property(s).

3. The method of claim 2, wherein the properties include a primary property and a secondary property.

4. The method of claim 3, wherein the secondary property score is a weighted score which includes both a primary metric score and an additional property score.

5. The method of claim 3, further comprising:
providing a target receptor, and the primary property is the binding affinity of the designed molecules to the target receptor.

6. The method of claim 5, further comprising posing the designed molecules in different poses with respect to the target receptor, and determining the binding affinity of the designed molecule with respect to each pose.

7. The method of claim 1, further comprising training a model using the known molecules; and
generating one or more new molecules using the trained model.

8. The method of claim 1, further comprising training a model using the known molecules and user provided molecules having known properties; and
generating one or more new molecules using the trained model.

9. The method of claim 7, further comprising generating one or more new molecules using the trained model using the known molecules of the first dataset and at least a portion of the molecules of the second dataset.

10. The method of claim 8, further comprising generating one or more new molecules using the trained model using the known molecules and the user provided known molecules of the first dataset and at least a portion of the molecules of the second dataset.

11. A iterative method for targeted molecular design comprising:
accessing a molecular database;
adding one or more head start molecules to a molecular database;
measuring the added one or more head start molecules against one or more metrics, including a primary metric and at least one secondary metric, wherein the metrics relate to at least one of the binding affinity of a molecule to a target receptor and an additional metric adding the measured one or more head start molecules to a master results table;
assigning one or more scores for each at least one secondary metric to the one or more head starthead start molecules in the master results table;
selecting one or more head start molecules based on the assigned scores for each of the primary metric, the at least one secondary metric, and a random molecule selected from the one or more head start molecules;
training a model using the selected one or more head start molecules; and
generating one or more generations of new molecules based on the trained model.

12. The method of claim 11, further comprising designating a first defined number of the head start molecules having the highest scores for the primary metric and using those first defined number of head start molecules having the highest scores for the primary metric as the selected one or more head start molecules for training the model.

13. The method of claim 12, further comprising additionally designating a second defined number of the head start molecules having the highest scores for the at least one secondary metric and using those second defined number of head start molecules having the highest scores for the at least one secondary metric as additional selected one or more head start molecules for training the model.

14. The method of claim 13, wherein the second defined number is less than the first defined number.

15. The method of claim 11, further comprising:
selecting a target receptor;
selecting a portion of at least one new molecule, and determining the binding affinity of the portion of the at least one new molecule to the target receptor.

16. The method of claim 15, further comprising posing the new molecule in different poses with respect to the target receptor; and
determining the binding affinity of the portion of the at least one new molecule to the target receptor in each pose.

17. The method of claim 12, further comprising:
after generating one or more new molecules based on the trained model as a first generation of new molecules, selecting the first defined number of new molecules from the first generation of new molecules, the first defined number of new molecules from the first generation of new molecules being those with the highest score against the primary metrics; and
generating a second generation of new molecules using the first defined number of new molecules with the trained model.

18. The method of claim 17, further comprising after generating one or more new molecules based on the trained model as a first generation of new molecules, selecting the second defined number of new molecules from the first generation of new molecules, the first defined number of new molecules from the first generation of new molecules being those with the highest score against the secondary metric; and
generating a second generation of new molecules using the first defined number of new molecules and the second defined number of new molecules with the trained model.

19. The method of claim 18, further comprising randomly selecting a head start molecule, and generating a second generation of new molecules using the first defined number of new molecules, the second defined number of new molecules, and the random molecule with the trained model.

20. The method of claim 19, further comprising displaying a table comprising each new molecule and the score thereof against the primary metric and the one or more secondary metrics.
